(19) United States
(12) Patent Application Publication
(10) Pub. No.: US 2014/0142427 A1
(43) Pub. Date: May 22, 2014

(54) Automated Fluid Delivery Catheter and System

(71) Applicant: LIGHTLAB IMAGING, INC., Westford, MA (US)

(72) Inventor: Christopher Petroff, Groton, MA (US)

(73) Assignee: LIGHTLAB IMAGING, INC., Westford, MA (US)

(21) Appl. No.: 13/792,910

(22) Filed: Mar. 11, 2013

Related U.S. Application Data
(60) Provisional application No. 61/727,320, filed on Nov. 16, 2012.

(51) Int. Cl. A61M 25/00; A61B 5/00

(52) U.S. Cl. CPC A61M 25/0021; A61B 5/0073
USPC 600/427; 604/122

(57) ABSTRACT

In part, the invention relates to catheters, methods, and blood clearing technologies suitable for use in an optical coherence tomography system. The optical coherence tomography system includes a control system, a probe including a catheter defining a lumen and a rotatable optical fiber located within the lumen, a fluid cartridge holder in communication with the lumen of the probe, a pump to move fluid from the fluid cartridge to the lumen of the probe; and a motor configured to rotate and pull the optical fiber through the lumen of a blood vessel. The pump and the motor are controlled by the control system. The catheter can include a wall that bounds the lumen of the probe, which defines a flush port and includes a valve in fluid communication with the flush port, the valve configured to permit fluid from the lumen to pass through the wall.

### CROSS-REFERENCE TO RELATED APPLICATIONS

This application claims priority to and the benefit of U.S. Provisional Patent Application No. 61/727,320, filed on Nov. 16, 2012, the entire disclosure of which is herein incorporated by reference.

### FIELD OF THE INVENTION

In part, the invention relates generally to blood clearing devices, related methods, components, and materials suitable for use with imaging systems such as optical coherence tomography data collection systems.

### BACKGROUND OF THE INVENTION

A probe used for optical coherence tomography (OCT) and other blood vessel intraluminal imaging modalities typically includes a catheter having a lumen constructed for use with an optical fiber. During imaging, the optical fiber is rotated within the lumen. Light from a light source is transmitted through the optical fiber, leaving the optical fiber at its distal end and passing through the optical catheter wall. As the optical fiber rotates, the light beam from the optical fiber sweeps and illuminates the blood vessel wall. Light reflected by the blood vessel wall is returned to the optical fiber again by passing through the wall of the optical catheter.

In turn, the light travels back through the optical fiber to an interferometer connected to the proximal end of the optical fiber. An interference pattern is generated when the light reflected by the vessel wall is combined with light from the interferometric light source. The pattern is then interpreted by a computer. The computer then generates an image of a portion of the blood vessel. Accordingly, by pulling an optical fiber through a blood vessel while the optical fiber is spinning, a three dimensional image of the blood vessel can be constructed.

More light will leave and enter the optical fiber if the refractive indices of the fluid inside the catheter lumen and the fluid in the blood vessel outside the catheter are matched. To achieve index matching, a fluid is typically introduced into the catheter lumen that more closely matches the fluid of the physiological site. Certain imaging modalities, including OCT, are degraded when imaging is attempted through an optical field containing blood cells. Methods exist for clearing blood from an optical field by flushing the optical field with fluid originating from the catheter lumen. However, maintaining a constant flow of fluid through the small diameter of the catheter while imaging a section of a blood vessel is difficult. This is especially true when blood clearing must be synchronized with image data collection.

Adding to the difficulty is the fact that assembling all the components used to flush a lumen takes time and makes the use of imaging modalities less likely to be adopted as a standard of care. Unfortunately, the lack of vessel information may result in sub-optimal treatments. For example, hand pumps and other modalities can be used to manually flush a lumen. However, many of these require a significant amount of set up time and sufficient strength and dexterity on the part of the clinician or operator. Manual systems can also be used to purge a catheter of air using a solution, such as contrast solution or other solution described herein, to prevent excess air from being introduced into the blood vessel. The imaging catheter is purged of air to reduce the risk of causing embolisms, or air bubbles, in the blood.

To understand the timeline and number of actions that are involved with manual purging of air, it is useful to consider a typical procedure, which has the following steps: (1) providing a container of contrast fluid; (2) filling a syringe with the contrast fluid; (3) removing air from the purge syringe by plunging the syringe until all air in the syringe is expelled; (4) attaching the syringe to the OCT catheter; (5) purging the OCT catheter by injecting contrast fluid into the catheter to remove air trapped in the catheter and (6) enabling the OCT system. Furthermore, the process must be repeated if the flush solution for clearing the blood vessel is different than the purge solution used to clear the catheter.

A need therefore exists for a system, apparatus, and method that improve image data collection by addressing problems caused by the presence of blood and other materials or particulates within the region being imaged. The present invention addresses this need and others and eliminates a number of these steps normally required by a typical OCT procedure by providing a rapid and elegant solution.

### SUMMARY OF THE INVENTION

In part, the invention relates to an optical coherence tomography system. In one embodiment, the system includes a data collection probe including: a catheter defining a lumen; and a rotatable optical fiber located within the lumen; a fluid cartridge holder in fluid communication with the lumen of the catheter; a pump positioned to be in mechanical communication with a fluid cartridge when the fluid cartridge is disposed in the fluid cartridge holder, the pump configured to move fluid from the fluid cartridge to the lumen of the data collection probe; a motor configured to pullback the optical fiber during data collection; and a control system configured to control one or both of the pump and motor. In another embodiment, the catheter has a wall that bounds the lumen. The wall defines a flush port and further includes a valve in fluid communication with the flush port, the valve configured to permit fluid from the lumen to pass through the wall. In yet another embodiment, the valve is an expandable tube valve. In still yet another embodiment, the valve opens in response to a pressure level of between about 50 psi and about 200 psi. In another embodiment, the wall defines a purge port configured to permit fluid from the lumen to displace air from the lumen.

In one embodiment, the data collection probe is at least one of an OCT probe, an ultrasound probe, or a pressure probe. In another embodiment, the control system is configured to signal the pump to stop pumping if insufficient fluid from the fluid cartridge has been delivered to permit data collection within a predetermined amount of time. In yet another embodiment, the control system is configured to signal the pump to stop pumping upon completion of the data collection. In still yet another embodiment, the fluid cartridge includes: a cartridge wall, a reservoir at least partially defined by the cartridge wall, a fluid disposed in the reservoir, and a fluid delivery port in fluid communication with the catheter lumen, whereby in response to fluid pressure, fluid moves from the reservoir through the fluid delivery port and into the catheter lumen, wherein the fluid pressure is triggered by the control system.

In another aspect, the invention relates to a catheter configured to clear blood in a vessel. In one embodiment, the catheter includes a catheter wall defining a first lumen; a probe located within the first lumen; a flush port defined by the catheter wall; a valve in fluid communication with the first lumen, the valve configured to permit fluid from the first lumen to pass through the catheter wall; and a purge port defined by the catheter wall and in fluid communication with the first lumen. The purge port is configured to permit fluid from the first lumen to purge air from the first lumen. In another embodiment, the valve includes an expandable tube. In yet another embodiment, the probe includes an optical fiber configured to slide and rotate relative to the catheter. In still yet another embodiment, the catheter wall defines a plurality of flush ports. In one embodiment, a first end of the expandable tube attaches circumferentially to the catheter wall. In another embodiment, the expandable tube covers the flush port and substantially seals the flush port when the expandable tube is in an unexpanded configuration. In yet another embodiment, the fluid delivery port is constructed to engage a fluid cartridge containing flush solution.

In another aspect, the invention relates to a method of collecting optical coherence tomography data. In one embodiment, the method includes triggering a purge to remove air from a catheter having a wall defining a lumen and defining a flush port and a purge port. The method further includes passing a flush solution through the lumen such that it exits the purge port and increasing pressure of the fluid flowing through the lumen to open the flush port. The method also includes triggering a motorized pullback configured to withdraw an optical fiber having a beam director from the vessel while collecting optical coherence tomography image data using the beam director while the fluid is leaving the flush port; and collecting optical coherence tomography image data during a portion of the pullback. In one embodiment, the method includes the step of pressurizing a fluid reservoir using a pump, the fluid reservoir in fluid communication with the catheter. In another embodiment, the method includes the step of stopping the pump after optical coherence tomography data has been collected. In yet another embodiment, the method includes the step of flushing a blood vessel.

### BRIEF DESCRIPTION OF DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views. The drawings associated with the disclosure are addressed on an individual basis within the disclosure as they are introduced.

FIG. 1a is a schematic diagram of a catheter and OCT data collection system in accordance with an embodiment of the invention.

FIG. 1b is an exemplary graph of motor position versus time for a flush cycle in accordance with an embodiment of the invention.

FIG. 1c includes two exemplary graphs of pressure versus location in an OCT catheter and a guide catheter, respectively in accordance with an embodiment of the invention.

FIG. 1d is an exemplary graph of motor position versus time for various phases associated with using an OCT probe in accordance with an embodiment of the invention.

FIG. 2 is a side view of a patient interface unit, reservoir and OCT probe in accordance with an embodiment of the invention.

FIGS. 3a-c are views of (FIG. 3a) a fluid cartridge, (FIG. 3b) a fluid cartridge chamber that receives the fluid cartridge, and (FIG. 3c) a fluid cartridge engaged in the fluid cartridge chamber, in accordance with embodiments of the invention.

FIG. 4a is a cross-sectional view of a flush valve in a closed state in accordance with an embodiment of the invention.

FIG. 4b is a cross-sectional view of a flush valve in an open state in accordance with an embodiment of the invention.

FIG. 4c is another view of an embodiment of a flush Valve®.

FIG. 5a is a plan view of a catheter and syringe prior to connection in accordance with an embodiment of the invention.

FIG. 5b is a cross-sectional view of the catheter and the syringe of FIG. 5a prior to connection in accordance with an embodiment of the invention.

FIG. 6a is a plan view of a catheter and syringe at the beginning of connection but after the septum of the syringe has been punctured in accordance with an embodiment of the invention.

FIG. 6b is a cross-sectional view of the catheter and syringe of FIG. 6a at the beginning of connection but after the septum of the syringe has been punctured in accordance with an embodiment of the invention.

FIG. 7a is a plan view of a catheter and syringe in connected arrangement in accordance with an embodiment of the invention.

FIG. 7b is a cross-sectional view of the catheter and the syringe of FIG. 7a in a connected arrangement in accordance with an embodiment of the invention.

FIG. 8 is a graph of viscosity versus concentration for various molecular weights of Dextran suitable for use with an embodiment of the invention.

### DETAILED DESCRIPTION

When OCT data is collected in a blood vessel, blood is initially dispersed around the probe that is used to collect data. An OCT scan in the presence of a blood field can result in the blood being misinterpreted as tissue. In part, this can occur because of the optical and interferometric nature of OCT. Therefore, it is desirable to clear blood from a blood vessel to improve the images of the vessel generated using data collected using an OCT probe. In one embodiment, a flush is used to clear a region of the blood vessel so that OCT imaging can be performed. Sections of the OCT probe can also have air disposed therein which is purged using a suitable solution prior to use in a subject. In part, embodiments of the invention relate to port placements, catheter configurations, flush and purge systems, components of the foregoing and related methods to expedite a given purge and/or flush procedure.

Various conveniences and devices for performing flushing and purging as part of the operation of an OCT system are described herein. For example, in part, the invention also relates to prefilled cartridges that can include a flush solution, and a purge solution or a solution suitable for both types of fluid delivery. In one embodiment, cartridges can be implemented as prefilled syringes. The cartridges can be placed in a holder configured to receive and/or orient each cartridge. These cartridges can be used with a purge and flush catheter system. In one embodiment, a cartridge is used to both purge a catheter and flush a blood vessel prior to imaging the blood vessel. In addition, cartridges can be used with a motorized catheter patient interface unit to automatically cause blood clearing during an OCT data collection session. Thus, a cartridge can be placed in a holder and caused to release its fluid contents in response to a trigger or event generated, used or relayed by a control system. Cartridges are designed to provide a time saving convenience and allow for sterile solutions to be easily stored. In addition cartridges further reduce preparation and operation time for a given purge or flush procedure.

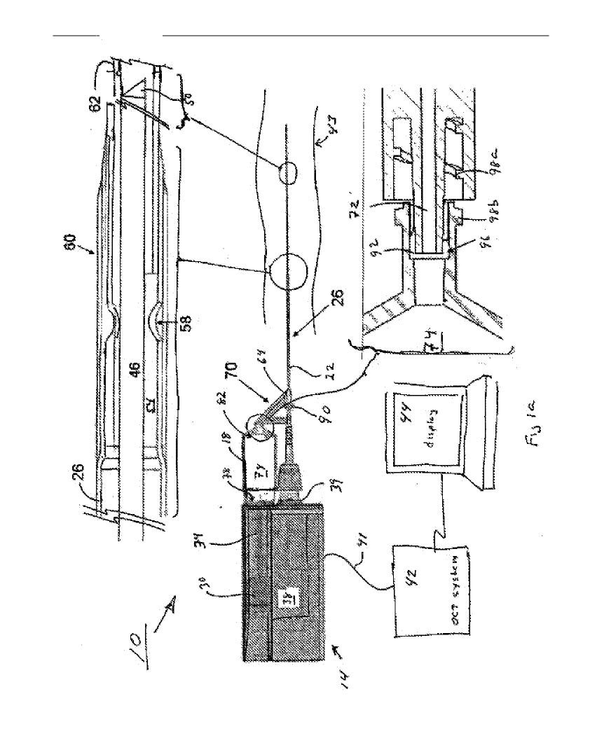

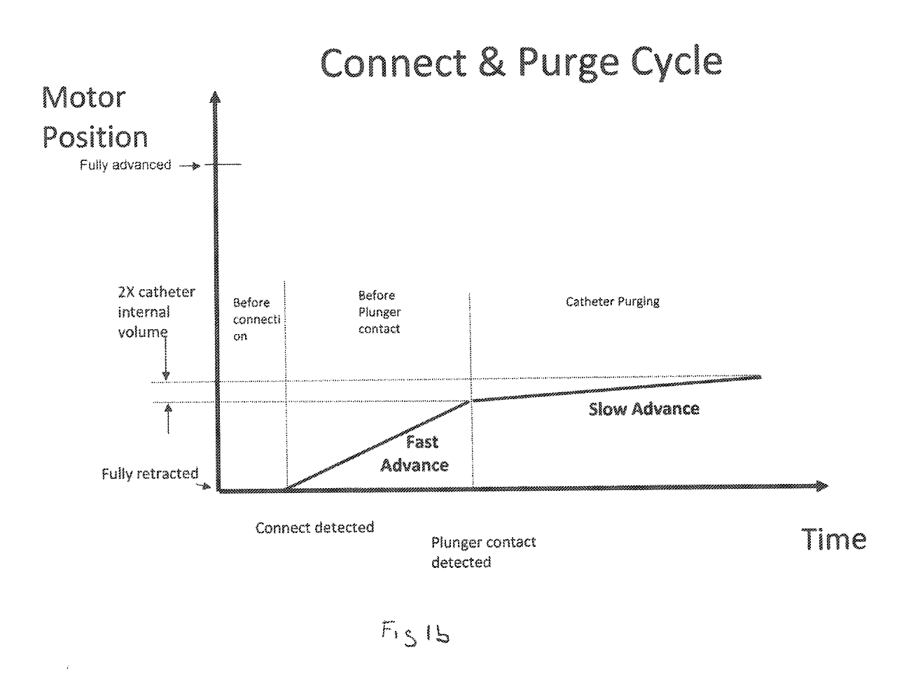

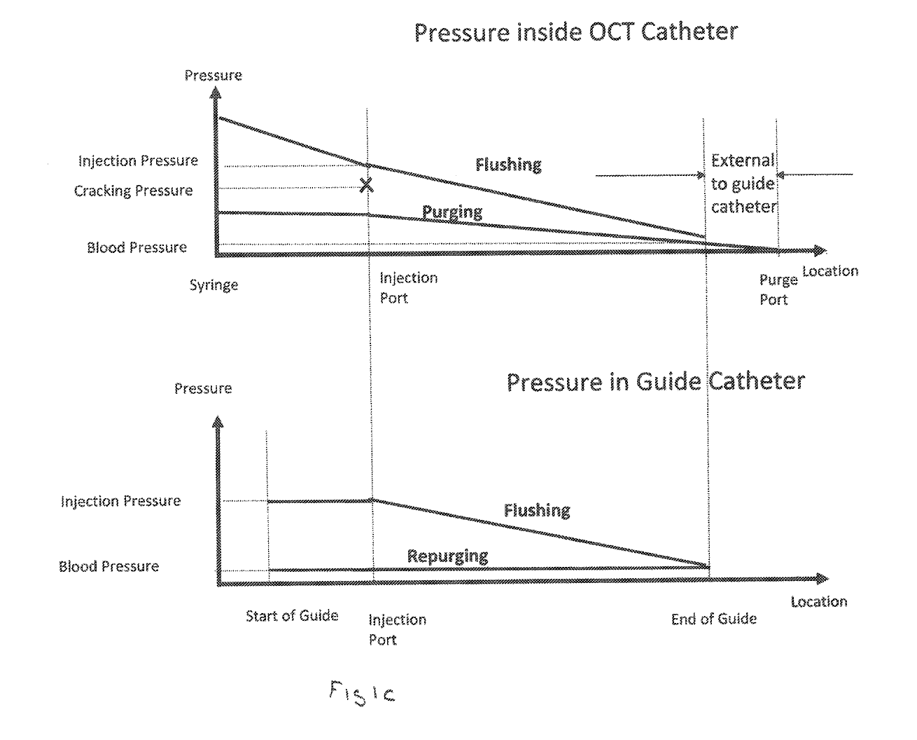

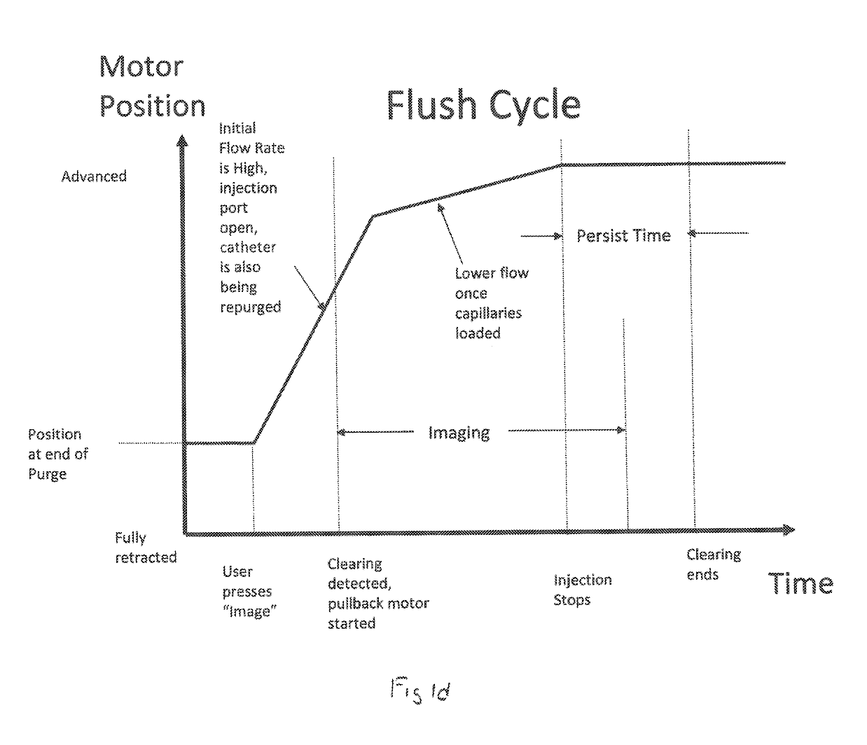

In brief overview, and referring to FIG. 1a, the system 10 includes a patient interface unit (PIU) 14, a fluid cartridge chamber (or holder) 18, a fluid cartridge 74, a guide catheter 22, an OCT probe 26 and an OCT interferometric and control system 42. In one embodiment, the OCT probe 26 includes an imaging catheter with an optical fiber disposed therein. During image data collection, the OCT probe 26 within the guide catheter 22 is positioned in a blood vessel 43 of a patient. The system 10 is configured to permit automated blood clearing that is safe and effective for blood vessel imaging. The fluid in the fluid cartridge 74 is used to purge the lumen of the catheter and clear blood from the lumen of a blood vessel. In one embodiment, the PIU 14 or a control system, such as the OCT interferometric and control system 42 or a component thereof, in communication with the PIU 14 controls the flow of fluid from the fluid cartridge 74 into the OCT probe 26. The PIU 14 can also control the rotational and longitudinal movement of the components of OCT probe 26 during the collecting of image data in a blood vessel. Light reflected from a vessel wall is received by the beam director 50 of OCT probe 26 and subsequently transmitted to the PIU 14 and then to the OCT system 42. In one embodiment, the fluid cartridge is a syringe or a portion thereof.

In one embodiment, the OCT probe 26 is introduced into a vessel of a patient within a guide catheter 22 through a Tuohy-Borst connector 90. In one embodiment, the flushing solution is injected proximally to the flush ports just after the Tuohy-Borst connector. In turn, the guide catheter 22 carries the flush solution to the area of the vessel where OCT data will be collected. Injecting the flush solution through the catheter directly eliminates the need for a separate fluid injection line in addition to all the other associated connections in the PIU interface.

The OCT interferometric and control system 42 constructs or generates one or more images of the vessel using the OCT image data reflected from the vessel wall. The OCT system 42 includes a data acquisition system or component that includes or is in electrical communication with a display 44. The display 44 is configured to display images of a blood vessel, e.g., cross-sectional and three-dimensional images. In one embodiment, the display 44 can be used to input control information to the system 42. The display can include a processor or the processor can be part of the OCT interferometric and control system 42. The processor can be used to analyze and process OCT data and generate control signals for the pump and/or motor in one embodiment.

In more detail, the PIU 14 includes one or more motors 30 or linear actuators to drive a piston 34 bi-directionally. Although in this embodiment a linearly driven piston is used, other embodiments are possible in which fluid is forced from a sterile container without using a piston. The PIU 14 also includes one or more motors 38 or rotational actuators that rotate an optical connector 39. Such an optical connector can be in communication both with the data acquisition portion of the OCT interferometric and control system 42 through optical fiber 41 and with an optical fiber 46 of the OCT probe 26.

Referring to FIG. 1b, the motor 30 initially is set so that the piston 34 is fully retracted before the fluid containing element such as fluid cartridge 74 is connected. Once the fluid cartridge 74, such as a syringe, is in place in a holder and the connection to the guide catheter 22 is made, the connection of the catheter with the PIU 14 is detected by the OCT system 42. The motor 30 is activated and automatically advances the piston 34 at a suitable speed, as part of the fast advance phase, to apply pressure to the fluid disposed in the reservoir defined by the wall of the cartridge 74. A torque sensor in the motor 30 detects when the piston 34 makes contact with a cartridge 74 such as a disposable syringe. In one embodiment, the disposable syringe can be supplied with the catheter 22 or supplied separately.

The motor 30 then slows down as shown by the slope of the line decreasing to a second speed corresponding to the slow advance phase. The initial fast speed is greater than the second speed used for the slow advance phase. The motor 30 continues to advance the piston 34 at this second speed, forcing liquid out of the syringe and into the catheter 22. The amount of liquid expelled is greater than the internal volume of the catheter 22. In one embodiment, this amount is twice the internal volume of the catheter. This excess fluid volume insures all the air is removed from the catheter lumen. This excess fluid volume eliminates the need for the operator to watch for liquid exiting the purge port as purging is insured by this operating mode.

Referring to FIG. 1c the pressures in the OCT probe 26 and in the guide catheter 22 are shown during different operational modes of the system. As shown in the “purging” line in the graph of the pressure inside the OCT catheter, during purging, the flow pressure is less than flow pressure during flushing and hence the flow is lower. The cracking or valve opening pressure of the flushing port is shown in FIG. 1c. During purging the advance speed is slow enough that the pressure at the syringe port is below cracking pressure of the flush port valve 58 in the catheter and hence the flush port valve 58 remains closed. The valve has what is termed zero dead volume, because the valve has no locations in which air can be trapped as the flush solution replaces the air inside the catheter.

FIG. 1d shows the piston 34 position as it changes during the blood vessel flushing cycle. The piston 34 begins at the position it was at when the purge was completed. When the operator wishes to begin to take imaging data, the operator starts the procedure and this causes the motor 30 to operate at a high speed, increasing the flow pressure and thereby opening the flush port valve 58. This results in re-purging the catheter thereby removing any blood that may have entered the catheter. As soon as the blood clearing is detected by the software monitoring the OCT image, the imaging fiber 46 is withdrawn by a pullback motor 38. Once about 3 to 5 ml of liquid is flushed through the catheter, the syringe motor 30 is slowed and the fluid injection rate is decreased.

The coronary arteries contain 2 to 3 ml of blood. Once the capillaries are loaded with a high viscosity flush solution, the flow rate can be reduced because the blood flow slows down in the coronary arteries. The bolus of 3 to 5 ml of liquid that is injected at the high rate causes the capillaries to be loaded with flush solution. However, if a flush solution is used that has a viscosity that matches blood, then the high initial flow rate is used for the entire flush cycle because the flow in the capillaries will not slow when a flush solution with a matching viscosity is used. One the flush is complete, the motor 30 is then stopped and fluid injection is terminated before the imaging stops because the vessel will remain cleared of blood for some time afterwards (“persist time”). When the persist time continues past the imaging stop time, the amount of liquid injected through the catheter is sufficient.

The pressure profiles during flushing are also depicted in FIG. 1c. That is, when flushing of the blood vessel is desired, the motor 30 moves the piston 34 faster, which increases the pressure at the cartridge/syringe and the cartridge/syringe port. This is shown by the “flushing” line in the Pressure Inside OCT Catheter graph of FIG. 1c. Since the pressure at the injection port is now higher than the cracking pressure of the flush port valve 60, flow will exit through the flush port 58. Whether purging or flushing, the pressure is always highest at the syringe and lowest at the purge port 62. When flushing, the imaging lumen of the catheter is automatically flushed of blood as the high pressure required to open the flushing port also re-purges the catheter of blood.

Should additional flush solution be needed for additional OCT imaging, the user removes the catheter from the patient, disconnects the catheter from the PIU, removes the empty syringe, attaches a new syringe and performs the cycle that is shown in FIG. 1b. Any air that may have been introduced during the reconnection of the syringe will be expelled by the new purge cycle. Thus there is no concern about introducing air into the patient when a syringe is swapped. The syringe is sized such that it would rarely need to be swapped during a normal procedure.

Further, in some embodiments of the OCT system, the user will select the vessel being imaged. If the vessel being imaged is on the right side as opposed to the left coronary arteries, the maximum coronary flow is less and consequently a slower flow rate may be used to flush the vessel. Other imaged arteries have different volumes between the imaged areas and the downstream capillaries. The initial high flow volume may be adjusted based on this volume. The flush fluid delivery rate may also be adjusted based on the size of the blood vessel, such as an artery, being imaged. A large artery will service more capillaries, which allows for more blood flow, and hence will require a larger amount of fluid be infused.

In one embodiment, the motors have encoders and a “home” switch to determine the position and angle of the probe and to control rotational and longitudinal speed of the probe. In some embodiments the motor includes a torque sensor that will shut or slow down the motor at certain torque levels. These preset torque levels provide a threshold that serves as a pressure limit. Instead of measuring pressure in the cartridge/syringe directly, the motor torque, which is correlated with measured pressures in the system, is used to determine a pressure measurement. Thus, a torque limit acts as a surrogate for a pressure limit.

Referring again to FIG. 1a, the OCT probe 26 defines a lumen 54 in which the optical fiber 46 is located. The OCT probe can be a multimodal probe such an OCT and ultrasound probe and any other probe that is configured to collect OCT data. Attached to the distal end of the optical fiber 46 is a beam director 50. Light that originates from an optical source in the OCT system 42 travels through optical connector 39. The light also travels along the optical fiber 46 before being directed through the wall of the OCT probe 26 by the beam director 50. The lumen 54 of the OCT probe 26 is in communication with the outside environment of the OCT probe 26. This communication can occur through both a purge port 62 and, under certain conditions, through a flush port 58 having a valve 60. The purge port 62 is an opening defined by the catheter wall. The catheter wall can include more than one purge port; for example, the catheter wall can define 2, 3, 4, 5, 6, 7, 8, or more purge ports.

The proximal end of the catheter 22 includes a fluid entry port 64. The fluid entry port 64 is connected to an outlet port of the fluid cartridge chamber 18 by way of a fluid channel 72 in connector 70. The fluid cartridge chamber 18 is sized and shaped to hold the removable fluid cartridge 74. In various embodiments, the system is configured to use a refillable fluid reservoir in fluid communication with the PIU or a disposable cartridge that can connect to the PIU. In the embodiment shown, a removable cartridge is compressed by the piston. However, any collapsible container that can have fluid pushed from it with a piston or otherwise acted upon to release its contents may be used. In the embodiment shown, the fluid injector is part of the PIU. In an alternative embodiment, the mechanical injector is separate from the PIU. The injector can be controlled by the same control line that controls the PIU or through another control line.

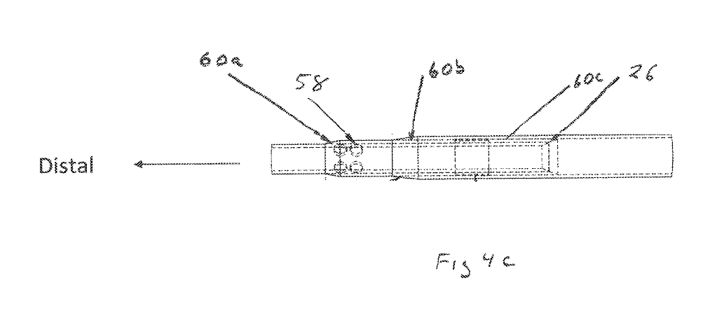

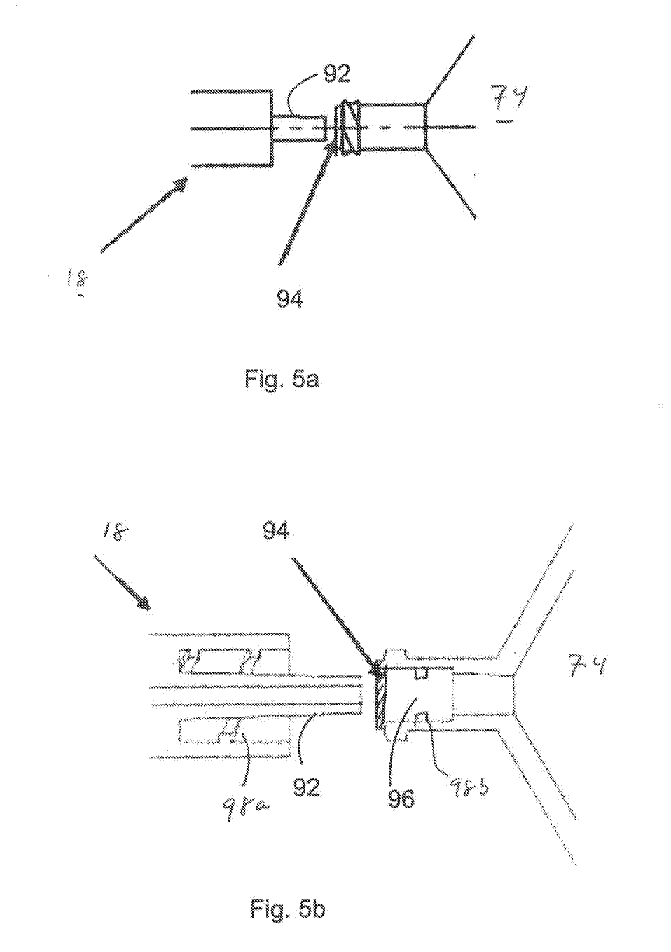

The fluid cartridge 74 contains a flush solution, preferably a sterile solution, having a predetermined viscosity and refractive index. In some embodiments, the proximal end 78 of the fluid cartridge 74 is in communication with the piston 34, while, in one embodiment, a septum at the distal end 82 of the fluid cartridge 74 is penetrated by an assembly positioned in the fluid cartridge chamber 18 (shown in more detail in FIGS. 5(a)-7(b)). The assembly permits the fluid disposed in the fluid cartridge 74 to enter the fluid channel 72 and pass into the lumen 54 of the OCT probe 26.

As the piston 34 is moved against the fluid cartridge, fluid is forced from the fluid cartridge 74 through the fluid channel 72 and into the lumen 54 of the optical probe. The faster the piston 34 is moved the higher the pressure that is applied to the flush solution disposed in the fluid cartridge 18. A torque sensor on the piston motor continuously monitors the pressure generated by the piston. Should the pressure exceed a preset limit, indicating the catheter is kinked, the piston motor 30 stops and the user is notified to check the catheter.

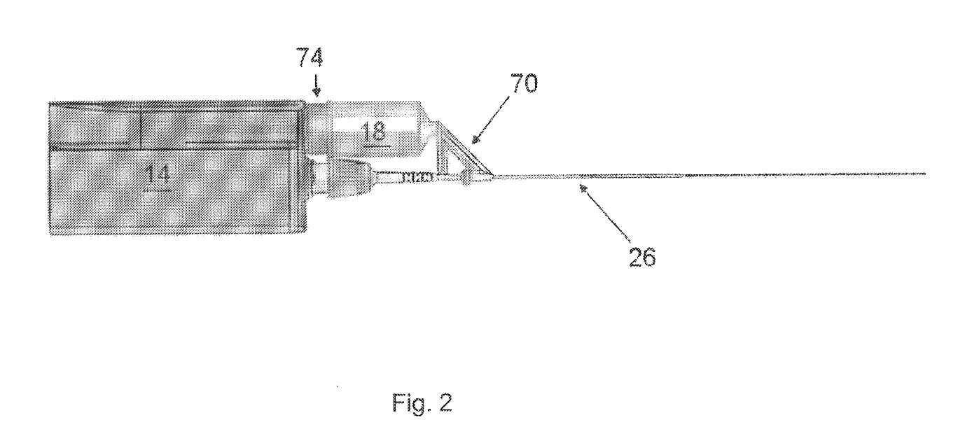

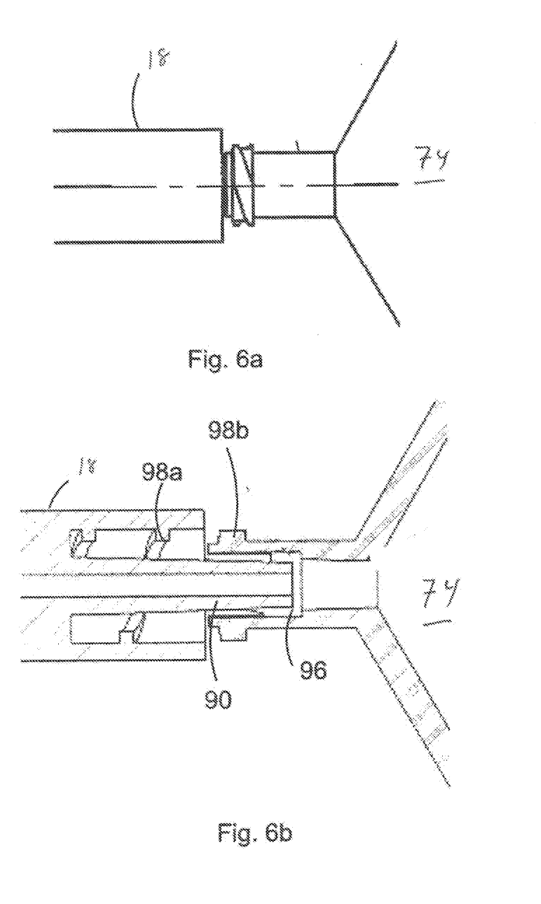

FIG. 2 is a side view of a fluid cartridge chamber 18 with fluid cartridge 74 and optical probe 26 installed on a PIU 14. The connector 70 can have an angled geometry as shown for ease of injection molding. A fitting 98a and 98b (FIG. 6) anchors the cartridge 74 and the cartridge chamber 18.

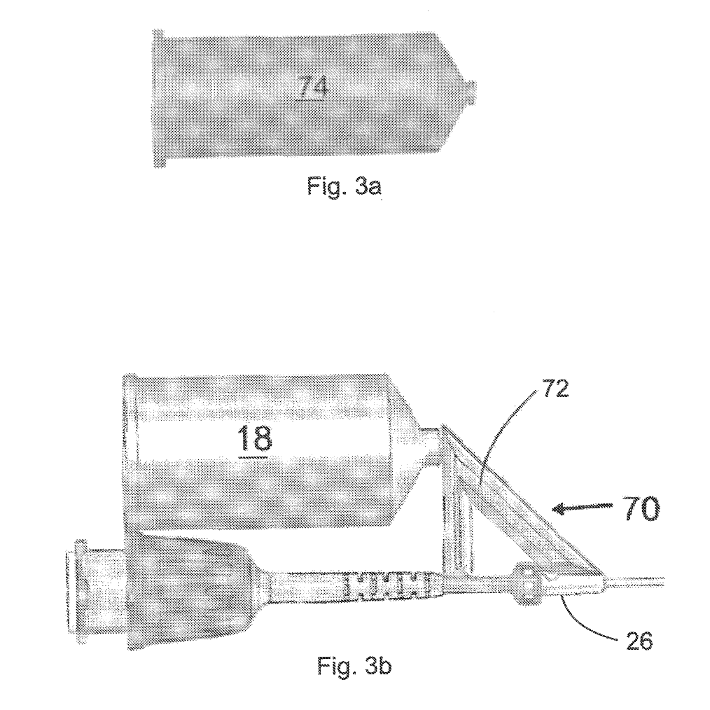

FIGS. 3a-c respectively depict an embodiment of a fluid cartridge 74 (FIG. 3a) and fluid cartridge chamber 18 (FIG. 3b) for receiving the fluid cartridge, which can be pre-assembled as a single unit (FIG. 3c) for mounting to the PIU 14. In one embodiment, the cartridge chamber 18 is held rigidly in position within the cartridge chamber 18. In one embodiment, a fluid channel 72 is located within the cartridge chamber 18.

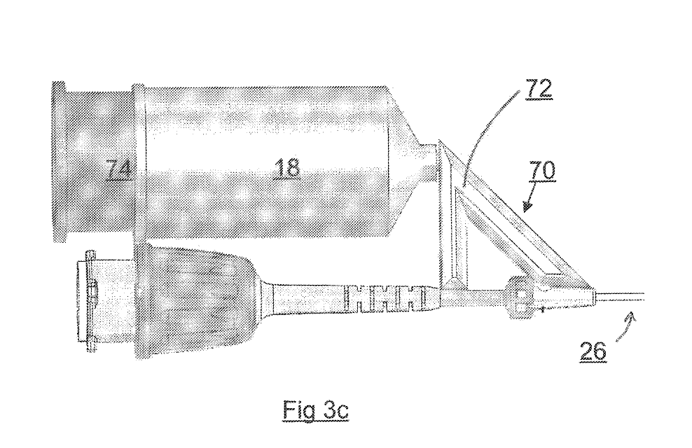

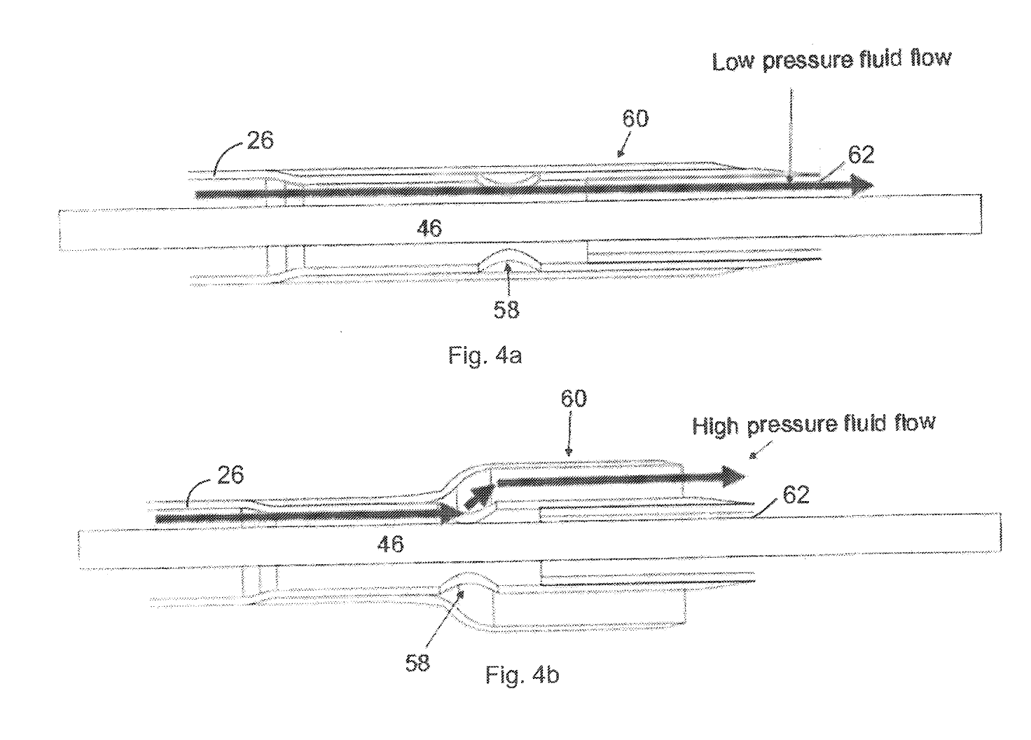

FIG. 4a depicts a view of the OCT probe 26 in the vicinity of a flush port 58 having a valve 60, such as an expandable tube valve, having a cracking or opening pressure. In some embodiments, a first end of the expandable tube valve 60 is attached circumferentially to the catheter wall and a second, distal end of the tube is unattached. The flexible tube valve or sheath 60 is positioned against the wall of the probe 26. The diameters of the ports 58 are large enough that the flush solution will displace any air in the ports 58. The expandable tube at least partially covers one or more flush ports 58. The proximal end of the valve 60 has a non-expandable band securing it to the outer wall of the probe 26. Alternatively that end of the valve 60 may be glued to the probe wall prior to the band being put into place. This band prevents the glue from fracturing when under pressure due to fluid flow (FIG. 4b). Under low pressure conditions, an expandable tube 60 is held by the resiliency of the tubular material against the flush port 58 keeping fluid in lumen 54 from passing through the flush port 58. In FIG. 4a, tube 60 is shown in a closed configuration.

Referring to FIG. 4c, in one embodiment, the expandable tube is made of three layers (60a, 60b, 60c) of Tecoflex® 80A (Lubrizol Corporation, Wickliffe, Ohio). Various other materials can be used to make the expandable tube in different embodiments. The thickness of the three layers is about 0.014″ while a single layer of Tecoflex is about 0.005″ thick. Tecoflex is attractive for this application because of its low modulus of elasticity (stiffness) by which the flush pressure can open it significantly without becoming permanently distorted. The pressure (P) to stretch the wall is given by:

P=2t(σ/D)

where:

t=wall thickness of Tecoflex;

σ=stress on tubing due to differences from its relaxed position;

D=inner diameter of the tube.

The expandable tube is stretched over the sheath which results in a cracking or opening pressure for the flexible tube valve. Below the cracking pressure there is no flow out of the sheath, and the stress, σ, on the stretched tube is:

σ=2E(i/D)

where:

E=modulus of elasticity of Tecoflex;

i=radial interference between the Tecfolex and the sheath which equals the unstretched Tecoflex radius minus the sheath radius; and σ and D are defined above.

By combining the two equations:

P=4t(E(i/D²))

Therefore when the flush pressure exceeds 4t(E(i/D²)), the tube will stretch. Tecoflex's E allows an acceptable P and t. When the tube is actually stretched open, modulus of elasticity E, increases due to Tecoflex's material properties. Thus the pressure required to stretch and hence open the valve wider is greater than the cracking pressure. A radial interference of about 0.002″ has been found acceptable to produce a reasonable cracking pressure.

In one embodiment, a low pressure condition refers to a pressure that ranges from about 0 to about 100 psi, and preferably between about 0 to about 50 psi. Under this condition, fluid only exits the lumen through the purge port 62.

FIG. 4b depicts a cross-section of the optical probe in the vicinity of the flush port 58 with the valve under higher pressure. Under higher pressure (e.g., between about 50 to about 200 psi, and preferably greater than about 100 psi) the tubular material 60 opens and is pushed away from the flush port 58 allowing fluid in lumen 54 to pass through the flush port 58.

The fluid used to flush the optical field, which is typically disposed in cartridge 74, is selected to have a viscosity sufficient to entrain and clear blood from the vessel. In one embodiment, the flushing solution comprises a fluid having a viscosity that ranges from about 4 to about 10 centipoise. In another embodiment, the flushing solution comprises a fluid having a viscosity of about 6 centipoise. In one embodiment, Dextran is used to flush the field to remove enough blood such that OCT data can be collected.

In still more detail FIG. 5a depicts the inside of the front of the cartridge chamber 18 having a protrusion 92. A fluid cartridge 74 having a septum 94 is also depicted. FIG. 5b is a cross sectional view of the inside of the front end of the cartridge chamber 18 and the fluid cartridge 74 shown in FIG. 5a. The front end of the cartridge chamber 18 is configured to interface with the fluid cartridge such that the septum 94 is pierced by protrusion 92. A channel 96 sized to receive the protrusion 92 is shown to the right of the septum 94 as part of a port of the disposable cartridge 74. The septum 94 allows the liquid in the cartridge 74 to remain sterile.

FIG. 6a depicts the front of the cartridge chamber 18 engaging the fluid cartridge 74. FIG. 6b is a cross-sectional view of the front of the cartridge chamber 18 engaging the fluid cartridge 74 shown in FIG. 6a. In this FIG. 6b it is seen that the protrusion 92 punctures the septum 94. The neck of the fluid cartridge 74 includes a channel 96 such as recess to allow the punctured septum 94 room to open.

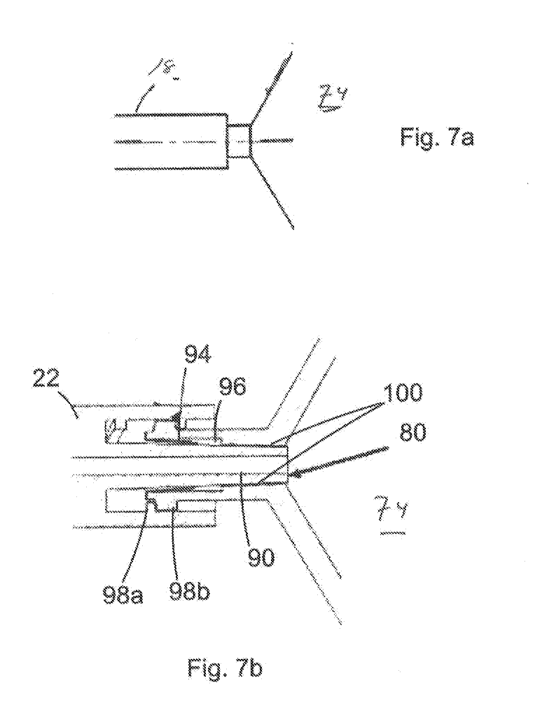

FIG. 7a depicts a fully engaged protrusion 92 and fluid cartridge 74. In FIG. 7b, a cross section of the catheter 22 and the sterile removable cartridge 74 shown in FIG. 4a is depicted. A cross sectional view of the locking mechanism 98a,b of the protrusion 92 holding the protrusion 92 in full engagement with the fluid cartridge 74 is shown in FIG. 7b. Luer type threads 98a on the protrusion locking mechanism 98a engage threads 98b on the fluid cartridge 74 and by screwing the cartridge threads and protrusion locking mechanism threads together, a leak-proof seal between the catheter and the fluid cartridge is formed.

As shown in FIG. 7b, the sterile removable cartridge 74 includes a sealing surface 100 and a septum 94 which retains the sterile fluid within the cartridge 74. The inside of the cartridge chamber 18 includes a protrusion 90 that is used to puncture the septum 94 and mate with the sealing surface 100 of the removable cartridge 74.

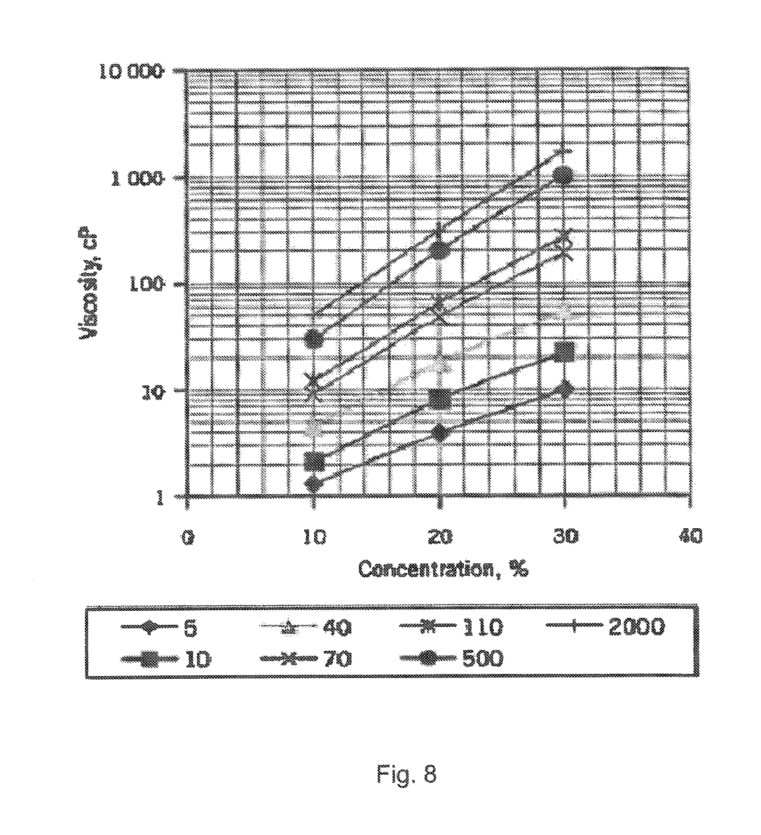

Referring to FIG. 8, the viscosity of various molecular weight Dextran concentrations is shown. Using a standard plasma expander intravenous 10% solution of low molecular weight Dextran 40 (average molecular weight 40,000 Daltons) in 5% dextrose (Hospira, Inc., Kale Forest, Ill. 60045 USA) the viscosity is about three centipoise, which can provides sufficient clearing of the blood vessel in certain scenarios. Other details relating to using Dextran flushes are described in U.S. Patent Pub. No. 20100076320, the disclosure of which is herein incorporated by reference in its entirety.

Although Dextran is described herein, any biologically compatible solution having viscosity in the range of about 4 to about 10 centipoise can be used. In one embodiment, the flush solution has a viscosity that ranges from about 3 cps to about 9 cps at body temperature. The flush solution can include a radio-opaque contrast solution. The contrast solution can include iodine having a concentration from about 150 mg/ml to about 400 mg/ml.

Returning to FIG. 1a, in operation, the fluid cartridge 74 is placed into the fluid chamber 18 and the fluid chamber 18 attached the PIU 14 such that the piston 34 comes in contact with the proximal end 78 of the fluid cartridge 74. The connector on the OCT probe 26 is simultaneously connected to the optical connector 39 of the PIU 14. The motor 30 is energized to force fluid from the fluid cartridge 74 into the lumen 54 and out through the purge port 62. This removes air from the lumen, which could otherwise cause an embolism and present a stroke or other risk to the patient. The motor 30 is controlled so as to properly purge the air without using an excessive amount of purging fluid. The use of the motor 30 also ensures that sufficient pressure is generated to permit the use of smaller diameter catheters such as a 5F catheter, which would be difficult to use with a hand driven syringe.

After purging the air from the lumen, the OCT probe 26 is then introduced into the guide catheter 22 which was previously positioned in the vessel of interest. The distal end of the OCT probe 26 is then moved to the region of interest in the blood vessel by activating the motor 38. In one embodiment, the optical connector 39 is configured to rotate at various speeds and to reduce vibration in the probe.

The optical connector 39 is rotated by motor 38 in response to a first command from the system 42 and the optical fiber 46 rotates. A second command to the PIU 14 from the OCT system 42 causes the optical fiber 46 to also be withdrawn from the blood vessel. At the same time as the optical fiber 46 is being withdrawn, the speed of a motor 30 is increased and the fluid expelled from the fluid cartridge 74 with higher pressure. The speed of the pump motor can range from about 2 ml/sec to about 8 ml/sec. This increased pressure (300 to 700 psi) causes the valve 60 of the flush port 58 to open and fluid to enter the space between the OCT probe 26 and the guide catheter 22 and out into the lumen of the blood vessel.

At this point blood, is cleared in a region of a vessel to be imaged. The clearing occurs as a result of the fluid exiting the catheter 22 through valve port 58. Once sufficient clearing occurs or after a predetermined period of time, OCT data collection is commenced. Typically, the flush uses 14 ml at a rate of flush of 4 ml/sec. The system 42 can detect if the flush has cleared the field within a given amount of time (for example 2.5 sec) and if not, the taking of OCT data is delayed. Alternatively the system can decide that the flush is not successful based on the amount of fluid required to clear the field rather than the amount of time needed to clear the field.

During this process, the OCT probe is typically pulled back through the blood vessel. Once the OCT imaging has been completed, the motor 30 is slowed again and the flush port valve 60 is automatically closed to prevent the unwanted expelling of fluid when from the optical probe 26 is removed from the guide catheter 22 and also to facilitate removal of the optical probe through the Touhy-Borst connector 90.

In the description above, embodiments of invention are discussed in the context of rotating imaging or forward scanning probes; however, these embodiments are not intended to be limiting and those skilled in the art will appreciate that the invention can also be used for other types of imaging applications, including non-biological applications.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be anyone of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

The aspects, embodiments, features, and examples disclosed herein are to be considered illustrative in an respects and are not intended to limit the invention, the scope of which is defined only by the claims. Other embodiments, modifications, and usages will be apparent to those skilled in the art without departing from the spirit and scope of the claimed invention.

What is claimed is:

1. An optical coherence tomography system comprising:
a data collection probe comprising
a catheter defining a lumen; and
a rotatable optical fiber located within the lumen;
a fluid cartridge holder in fluid communication with the lumen of the catheter;
a pump positioned to be in mechanical communication with a fluid cartridge when the fluid cartridge is disposed in the fluid cartridge holder, the pump configured to move fluid from the fluid cartridge into the lumen of the catheter
a motor configured to pullback the optical fiber during data collection; and
a control system configured to control one or both of the pump and motor.

2. The optical coherence tomography system of claim 1 wherein the catheter has a wall that bounds the lumen, the wall defining a flush port and further comprising a valve in fluid communication with the flush port, the valve configured to permit fluid from the lumen to pass through the wall.

3. The optical coherence tomography system of claim 2 wherein the valve is an expandable tube valve.

4. The optical coherence tomography system of claim 3 wherein the valve opens in response to a pressure level of between about 50 psi and about 200 psi.

5. The optical coherence tomography system of claim 1 wherein the wall defines a purge port configured to permit fluid from the lumen to displace air from the lumen.

6. The optical coherence tomography system of claim 1 wherein the data collection probe is at least one of an OCT probe, an ultrasound probe, or a pressure probe.

7. The optical coherence tomography system of claim 1, wherein the control system is configured to signal the pump to stop pumping if insufficient fluid from the fluid cartridge has been delivered to permit data collection within a predetermined amount of time.

8. The optical coherence tomography system of claim 1 wherein the control system is configured to signal the pump to stop pumping upon completion of OCT data collection.

9. The optical coherence tomography system of claim 1 further comprising:
the fluid cartridge wherein the fluid cartridge comprises:
a cartridge wall,
a reservoir at least partially defined by the cartridge wall,
a fluid disposed in the reservoir, and
a fluid delivery port in fluid communication with the lumen, whereby in response to fluid pressure, fluid moves from the reservoir through the fluid delivery port and into the lumen, wherein the fluid pressure is triggered by the control system.

10. A catheter configured to clear blood in a vessel comprising:
a catheter wall defining a first lumen;
a probe located within the first lumen;
a flush port defined by the catheter wall;
a valve in fluid communication with the first lumen, the valve configured to permit fluid from the first lumen to pass through the catheter wall; and
a purge port defined by the catheter wall and in fluid communication with the first lumen, the purge port configured to permit fluid from the first lumen to purge air from the first lumen.

11. The catheter of claim 10 wherein the valve comprises an expandable tube.

12. The catheter of claim 10 wherein the probe comprises an optical fiber configured to slide and rotate relative to the catheter.

13. The catheter of claim 10 wherein the catheter wall defines a plurality of flush ports.

14. The catheter of claim 10 wherein a first end of the expandable tube attaches circumferentially to the catheter wall.

15. The catheter of claim 10 wherein the expandable tube covers the flush port and substantially seals the flush port when the expandable tube is in an unexpanded configuration.

16. The catheter of claim 10 comprising a fluid delivery port for engaging a fluid cartridge containing flush solution.

17. A method of collecting optical coherence tomography data comprising:
triggering a purge of air from a catheter having a wall defining a lumen, the wall defining a flush port and a purge port;
passing flush solution through the lumen such that the flush solution exits the purge port;
increasing pressure of the fluid flowing through the lumen to open the flush port;
triggering a motorized pullback configured to withdraw an optical fiber having a beam director from the vessel while collecting optical coherence tomography image data using the beam director while the fluid is leaving the flush port; and
collecting optical coherence tomography image data during a portion of the pullback.

18. The method of claim 17 comprising pressurizing a fluid reservoir using a pump, the fluid reservoir in fluid communication with the catheter.

19. The method of claim 18 comprising stopping the pump after optical coherence tomography data has been collected.

20. The method of claim 17 comprising flushing a blood vessel.
